(19) United States
(12) Patent Application Publication
(10) Pub. No.: US 2006/0003015 A1
(43) Pub. Date: Jan. 5, 2006

(54) Food composition effective for treatment of oral and throat diseases and method of producing the same

(76) Inventor: Jae-joon Lee, Masan-si (KR)

Correspondence Address: Mark G. Lappin;McDermott Will & Emery LLP, 28 State Street, Boston, MA, 02109, US

(21) Appl. No.: 11/103,309

(22) Filed: Apr. 11, 2005

(30) Foreign Application Priority Data
Jul. 1, 2004 (KR) P2004-0051076

(51) Int. Cl. A61K 35/64; A61K 35/78; A61K 33/06

(52) U.S. Cl. 424/538; 424/698; 424/725.1

(57) ABSTRACT

The present invention relates to a food composition effective for the treatment of oral and throat diseases and a method of producing the same, and more particularly to a food composition effective for the treatment of oral and throat diseases, made of crude drug materials exhibiting no side effects to the human body and a method of producing the same. The food composition in accordance with the present invention comprises 25 to 45% by weight burnt alum, 0.001 to 5% by weight croton and 55 to 75% by weight gallnut, and thereby can provide excellent antibacterial and anticancer effects for oral microorganisms and mouth squamous cell carcinoma cell lines KB.

### BACKGROUND OF THE INVENTION

1. Field of the Invention

The present invention relates to a food composition effective for the treatment of oral and throat diseases and a method of producing the same, and more particularly to a food composition effective for the treatment of oral and throat diseases, made of crude drug materials exhibiting no side effects to the human body and a method of producing the same.

2. Description of the Related Art

Recently, due to changes in lifestyles and living environments, the consumption of sugars and a weakening of immunological functions due to stress has increased, and thus incidences of oral diseases have become increasingly higher. Development of oral diseases may be due to factors such as hosts, pathogens and the environment. When these factors are in a state of imbalance, there is a high possibility of development of certain oral diseases. Among the aforementioned factors, in particular microorganisms present in the oral cavity have been reported as the main cause of oral disease. Further, an increased incidence of oral cancer has been recently reported together with oral diseases due to oral microorganisms.

For these reasons, a number of attempts have been made to find materials effective for preventing and treating oral diseases using crude drugs or natural materials having no side effects on the human body. One attempt, Korean Patent Application Laid-Open No. 2003-0063914, in the name of the present inventor, discloses a method of producing a composition of a therapeutic material for oral and throat diseases, involving mixing and heating 3 to 4.5% by weight alum and 0.2 to 1.25% by weight croton at a temperature of 70 to 120° C. for 10 to 15 min to dissolve the alum; heating the dissolved alum at a temperature of 70 to 120° C. for 2 to 3 hours to evaporate water so as to solidify the alum; removing the croton from the solidified alum; mixing 3 to 4.5% by weight gallnut and 0.85 to 1.85% by weight indigo persicaria (Persicaria tinctoria H. Gross) into the solidified alum; and pulverizing the mixture into a powder.

However, the above-mentioned conventional method has weak antibacterial effects and further has no anticancer effect, and thereby it was not possible to use it as a composition for treating oral squamous cell carcinoma. Thus, there remains an urgent need for a food composition effective for the treatment of oral and throat diseases, exhibiting antibacterial effects, and further anticancer effects for the treatment of oral squamous cell carcinoma cell-line KB.

### SUMMARY OF THE INVENTION

Therefore, the present invention has been made in view of the above problems, and it is an object of the present invention to provide a food composition effective for the treatment of oral and throat diseases, exhibiting superior antibacterial effects, and further anticancer effects on oral squamous cell carcinoma cell-line KB, made of crude drug components; and a method of producing the same.

In accordance with the present invention, the above and other objects can be accomplished by the provision of a food composition effective for the treatment of oral and throat diseases, comprising 25 to 45% by weight burnt alum, 0.001 to 5% by weight croton and 55 to 75% by weight gallnut.

Preferably, the composition effective for the treatment of oral and throat diseases is in the form of powder.

In accordance with another aspect of the present invention, there is provided a method of producing a food composition effective for the treatment of oral and throat diseases, comprising the steps of:
mixing and heating 35 to 55% by weight alum and 0.1 to 15% by weight croton to dissolve the alum;
heating the dissolved alum to evaporate water so as to solidify the alum;
removing croton remnants from the solidified alum;
mixing the solidified alum with 35 to 55% by weight gallnut; and
pulverizing the mixture to obtain a powder thereof.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a process flow schematically illustrating production of a food composition effective for the treatment of oral and throat diseases in accordance with the present invention; and

FIG. 2 is a series of micrographs showing the results observed after cell viability and morphological changes in oral squamous cell carcinoma KB cell lines were tested using a food composition effective for the treatment of oral and throat diseases in accordance with the present invention in Example 3.

### DESCRIPTION OF THE PREFERRED EMBODIMENTS

Now, the present invention will be described in more detail.

First, the present invention provides a food composition effective for the treatment of oral and throat diseases. This composition includes 25 to 45% by weight burnt alum. The source material for the alum is alunite. The alum is prepared by removing foreign ingredients from the alunite, dissolving it in water followed by filtration, and boiling down the filtrate to obtain a crystal. This crystal, i.e., “alum,” is a colorless, odorless, transparent, and glossy material that has an astringent taste. Alum may be used internally or externally, and is easily dissolved in water, but not in alcohol. For internal use, it is sometimes used in its natural state, while for most medical applications burnt alum is prescribed. “Burnt alum” is a product obtained by heating alum to evaporate crystallized water contained therein. Burnt alum has potent antibacterial and preservative actions, and thereby is utilized in treating sore throat and tonsillitis. Further, for people suffering from frequent cold-sores, application of burnt alum to the affected area may have beneficial effects.

The inventive composition further includes 55 to 75% by weight gallnut. The gallnut is an insect gall produced by parasitism of Schlechtendalia chinensis, belonging to the family Aphididae of the order Hemiptera, on fresh leaves of Rhus javanica L. belonging to the family Anacardiaceae. Gallnut is known to have a cool and non-toxic nature and a sour and astringent taste. It is known that gallnut has an astriction effect due to its strong, puckery taste and thereby internal use thereof may treat chronic cough and diarrhea, and external use thereof may exhibit effects on various surgical and cutaneous diseases due to moist heat conditions, scabies and intractable wounds. Therefore, gallnut has efficacy in treating prolonged cough, prolonged dysentery and diarrhea, proctoptosis, heavy sweating of unknown cause, night sweats, ganacratia, anemia, epistaxis, metrorrhagia, traumatic bleeding, furuncular poisoning, thin skin abscesses, stomatitis, and the like. In addition, since tannic acid contained in gallnut has an action of precipitating proteins, when the tannic acid contacts mucosa ulcer of skin, the tissue proteins are immediately solidified, thus forming a film and thereby astriction is exhibited.

The composition in accordance with the present invention further includes 0.001 to 5% by weight croton. The croton (Croton tiglium L.), also referred to as croton seed in Chinese medicine, is an evergreen tree belonging to family Euphorbiaceae of order Geraniales and is used as a drastic. Croton contains a large amount of oil, which has significant harmful effects on the human body, and thus such oil must be removed before it can be used as a medicine. When the croton is heat-treated together with the alum, fatty components are removed therefrom and neutralized by the alum, as described below. The croton from which the poisonous component has been removed alleviates colic, throat edema, and sore throat.

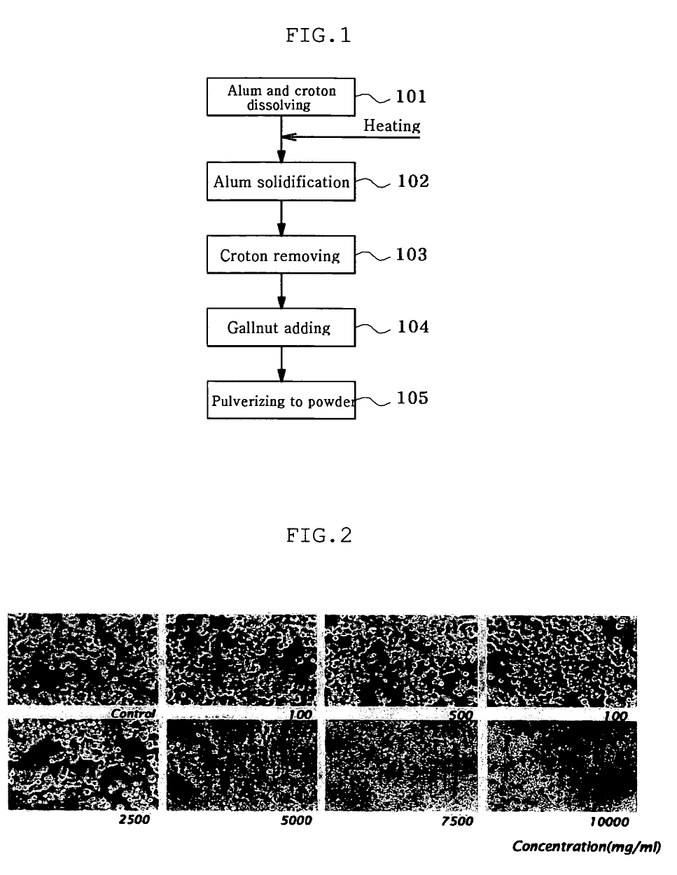

Further, the present invention provides a method of producing a food composition effective for the treatment of oral and throat diseases. FIG. 1 schematically shows a process flow for producing the composition in accordance with the present invention. The process comprises the steps of dissolving the alum and croton, solidifying the alum, removing croton remnants therefrom, mixing with gallnut and pulverizing the mixture.

In step 101, dissolving the alum and croton, 35 to 55% by weight alum and 0.1 to 15% by weight croton are mixed and heated to dissolve the alum. Preferably, heating and dissolving are preformed at a temperature of 70 to 120° C. for 10 to 15 min. In step 102, solidifying the alum, the dissolved alum is preferably heated to a temperature of 70 to 120° C. for 2 to 3 hours to evaporate water so as to solidify the alum. In steps 101 and 102, as fatty components are removed during the heat treatment process to solidify the alum, toxicity of the croton is neutralized with the alum, thus toxicity being eradicated. At this time, interaction between the alum and croton generates therapeutic ingredients from the alum, such as astringent, expectorant, hemostatic, and anti-ulcer components, and pharmacological ingredients from the croton, such as those effective for alleviating colic, throat edema and sore throat. Thus, having passed through the aforementioned steps, burnt alum is formed.

In step 103, removing the croton remnants, the croton remnants are removed from the solidified alum, i.e., burnt alum. Since the pharmacological ingredients of croton produced in steps 101 and 102 are infiltrated into the burnt alum, unnecessary residue of croton is removed in step 103.

In a mixing step 104, 35 to 55% by weight purified gallnut is added to the solidified alum, i.e., the burnt alum.

In step 105, pulverizing the mixture, the mixture of the burnt alum from which the croton remnants were removed is pulverized, along with the gallnut.

Now, the present invention will be described in more detail with reference to the following examples. These examples are provided only for illustrating the present invention and should not be construed as limiting the scope and sprit of the present invention.

### EXAMPLES

### Example 1

Preparation of a Food Composition Effective for the Treatment of Oral and Throat Diseases

3.75 g of alum and 0.25 g of croton were mixed and heated to a temperature of 70 to 120° C. for 10 to 15 min to dissolve the alum. Further heating was performed at a temperature of 70 to 120° C. for 2 to 3 hours until water contained in the alum was evaporated and the alum was solidified, so as to obtain the burnt alum, into which the croton ingredients were dissolved and infiltrated therein. The remnants of croton were removed from the burnt alum, and then 3.75 g of well-dried gallnut was mixed therewith. The mixture was pulverized to obtain a food composition effective for the treatment of oral and throat diseases (hereinafter, referred to as “composition A”) in powdered form.

### Example 2

Test of Antibacterial Effect on Microorganisms Isolated from Oral Cavity

Using composition A prepared in Example 1, an experiment to determine the antibacterial effects thereof was performed. The representative microorganisms relating to oral and bronchial inflammations, S.mutans(KCTC 3065), C. albicans(KCTC 7965), E.aerogenes(KCTC 2190), S.aureus(KCTC 1621) and E. coli(KCTC 1039), which are available from the Korea Collection for Type Cultures (KCTC) under the Korea Research Institute of Bioscience & Biotechnology (KRIBB, Daejeon, Korea), were used as test microorganisms. Each of the cultured test strains was taken in one loop, inoculated onto a liquid medium for the respective strains, and incubated at a temperature of 37° C. for 24 hours. 0.1 ml of the culture thus obtained was inoculated onto Muller Hinton liquid medium, incubated for 18 hours, and uniformly smeared on the Muller Hinton agar medium using a sterile swab. A 0.8 mm filter paper disc was placed on the medium in which the respective test strain was smeared and incubated at a temperature of 37° C. for 24 hours. After that, the antibacterial activity was observed as clear zones (mm) appearing around the disc.

TABLE 1
Inhibition Zone (mm)

| | E. | E. | S. | S. | C. |
|---|---|---|---|---|---|
| Samples/Strains | coli | aerogenes | aureus | mutans | albicans |
| Composition | 12 | 15 | 18 | 12 | 10 |
| A (powder) | | | | | |

As can be seen from Table 1, even when the composition in accordance with the present invention was used in powdered form, inhibition zones of 12, 15, 18, 12 and 10 mm, respectively, were observed, thus confirming excellent antibacterial activity.

### Comparative Example 1

The same procedure as in Example 1 was performed to prepare a composition (hereinafter, referred to as “composition B”), except that 1.25 g of indigo persicaria (Persicaria tinctoria H. Gross) was added in the step of mixing gallnut, in addition to 3.75 g of alum, 0.25 g of croton and 3.75 g of gallnut. The antibacterial effects of composition B were determined according to the same method as in Example 2. At this time, composition B was divided into a powder group and an extract group, in which the powder was dissolved in water; antibacterial effects thereof were then determined for the respective groups. The results thus obtained are shown in Table 2.

TABLE 2
Inhibition Zone (mm)

| | E. | E. | S. | S. | C. |
|---|---|---|---|---|---|
| Samples/Strains | coli | aerogenes | aureus | mutans | albicans |
| Composition B | — | — | — | — | — |
| (powder) | | | | | |
| Composition B | 9 | 9 | 9 | — | — |
| (extract) | | | | | |

As can be seen from Table 2, where composition B was used in powdered form, the inhibition zones were not observed at all, while where the water-extract of the composition B was used, insignificant inhibition zones were observed in E. coli, E.aerogenes and S.aureus, and further no inhibition zone was observed in S.mutans and C. albicans.

### Example 3

Anticancer Activity Test

Mouth carcinoma cell KB, which is available from the Korea Cell Line Bank (KCLB, Seoul, Korea), was used in this experiment by incubating it on RPMI 1640 medium containing 10% FBS, 100 U/ml penicillin-streptomycin.

To evaluate cell viability, composition A was added to be 100 μg/ml, 250 μg/ml and 500 μg/ml to the RPMI 1640 cultures, respectively. These cultures containing different concentrations of composition A were incubated for 24 hours and recovered, then Trypan blue was added to 0.2% concentration thereto followed by counting the number of viable cells using a hemocytometer. Average value of the number of viable cells and standard deviation were calculated by performing determination three times for every calculation. Further, changes in cell morphology were observed by light microscopy (MPS-30, Leica, Germany).

As can be seen from the results in FIG. 2, in case of composition A, reduction of cell viability was observed at a high concentration thereof.

As apparent from the above description, even with use of the composition in accordance with the present invention in powdered form, there are provided excellent antibacterial effects against E. coli, E.aerogenes, S.aureus, S.mutans and C. albicans, and also anticancer effects against mouth squamous cell carcinoma cell line KB.

Therefore, when used in treating a variety of oral diseases including intractable diseases such as oral inflammation, tongue fur, ulcers of the tongue, oral cancer, cancer of the tongue, acute/chronic tonsillitis and various incarnations of sore throat, the composition of the present invention has no side effects at all and exhibits excellent therapeutic effects.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A food composition effective for the treatment of oral and throat diseases, comprising 25 to 45% by weight burnt alum, 0.001 to 5% by weight croton and 55 to 75% by weight gallnut.

2. The food composition as set forth in claim 1, wherein the composition is in the form of powder.

3. A method of producing a food composition effective for the treatment of oral and throat diseases, comprising the steps of:
mixing and heating 35 to 55% by weight alum and 0.1 to 15% by weight croton to dissolve the alum;
heating the dissolved alum to evaporate water so as to solidify the alum;
removing croton remnants from the solidified alum;
mixing the solidified alum with 35 to 55% by weight gallnut; and
pulverizing the mixture to obtain powder thereof.

4. The process as set forth in claim 3, wherein the step of dissolving the alum is performed at a temperature of 70 to 120° C. for 10 to 15 min.

5. The process as set forth in claim 3, wherein the step of heating the dissolved alum to evaporate water so as to solidify the alum is performed at a temperature of 70 to 120° C. for 2 to 3 hours.
